(19) United States
(12) Patent Application Publication
(10) Pub. No.: US 2026/0165800 A1
(43) Pub. Date: Jun. 18, 2026

(54) REMOTE CONTROL FOR A SURGICAL ROBOTIC DEVICE

(71) Applicant: Microbot Medical Ltd., Yokneam Illit (IL)

(72) Inventors: Simon SHARON, Zichron Yaacov (IL); Idan BOADER, Karmiel (IL); Oren COHEN, Kadima (IL); Eran COHEN, Kiryat-Tivon (IL); Eyal MORAG, Tel Aviv (IL)

(73) Assignee: Microbot Medical Ltd., Yokneam Illit (IL)

(21) Appl. No.: 18/711,896

(22) PCT Filed: Nov. 18, 2022
(86) PCT No.: PCT/IL2022/051232

§ 371 Date: May 21, 2024

Related U.S. Application Data
(60) Provisional application No. 63/283,617, filed on Nov. 29, 2021.

(51) Int. Cl. A61B 34/37; A61B 17/00; A61B 34/35; A61B 90/00

(52) U.S. Cl. CPC A61B 34/37; A61B 17/00; A61B 34/35; A61B 90/37; A61B 2017/00212; A61B 2017/0042

(57) ABSTRACT

Systems and methods are provided for controlling, using a remote control, movement of first, second and third elongate surgical tools by a robotic device. The method involves controlling movement of the first and second elongate surgical tools via dedicated right and left button modules configured on the remote control; continuously holding a tool transition button configured on the remote control to control movement of the third elongate surgical tool using one of the right or left button modules; and then releasing the tool transition button to return to control of the first and second elongate surgical tools via the right and left button modules.

### RELATED APPLICATION/S

This application claims the benefit of priority of U.S. Provisional Patent Application No. 63/283,617 filed on Nov. 29, 2021, the contents of which are incorporated herein by reference in their entirety.

### FIELD AND BACKGROUND OF THE INVENTION

The present invention, in some embodiments thereof, relates to a remote control for use with a surgical robotic device, and, more particularly, but not exclusively, to control of multiple (e.g. more than 2) elongate surgical tools moveable by a robotic device via a remote control held and operated by two hands of a user.

PCT publication WO 2021/011571 to Clark et al. discloses “A system for controlling a catheter-based procedure system that includes a robotic drive configured to control rotational motion and axial motion of one or more elongated medical devices may include a body, a first control coupled to the body, and a second control coupled to the body. First control is configured to instruct the robotic drive to axially move one of the one or more elongated medical devices in response to manipulation of the first control by a user, and the second control is configured to instruct the robotic drive to rotate one of the one or more elongated medical devices in response to manipulation of the second control by the user, wherein the first control and the second control are positioned on the body so the first control and the second control can be simultaneously manipulated by a first digit and a second digit on a hand of the user.”

### SUMMARY OF THE INVENTION

According to an aspect of some embodiments there is provided a method of controlling, using a remote control, movement of first, second and third elongate surgical tools by a robotic device, the method comprising:
controlling movement of the first and second elongate surgical tools via dedicated right and left button modules configured on the remote control;
continuously holding a tool transition button configured on the remote control to control movement of the third elongate surgical tool using one of the right or left button modules; and
releasing the tool transition button to return to control of the first and second elongate surgical tools via the right and left button modules.

In some embodiments, the method comprises holding the remote control between the left and right palms of the user and operating the right and left button modules using the thumbs.

In some embodiments, the right and left button modules control movement of the first and second elongate surgical tools by default.

In some embodiments, the first, second and third elongate surgical tools include a guidewire, a microcatheter, and a guiding catheter which are telescopically arranged at the robotic device.

In some embodiments, controlling movement comprises one or both of rolling the elongate surgical tool, and linearly advancing or retracting the elongate surgical tool.

In some embodiments, controlling movement comprises rolling and linearly advancing or retracting the elongate surgical tool simultaneously, using the same control button on the remote control.

In some embodiments, controlling movement comprises increasing a speed of movement the elongate surgical tool.

In some embodiments, controlling movement comprises simultaneously moving two of the first, second and third elongate surgical tools.

In some embodiments, the method further comprises detecting gripping of the remote control between both of the user's hands, and allowing the controlling only if gripping is detected. In some embodiments, the method further comprises viewing a screen of an imaging modality while at the same time controlling the remote control.

In some embodiments, controlling movement via the remote control is performed at a different room than the surgical room in which the robotic device is located.

In some embodiments, when the tool transition button is continuously held, a function of at least some of the buttons of the right or left module changes.

In some embodiments, when the tool transition button is continuously held, an extent of movement enabled by at least some of the buttons of the right or left module changes.

In some embodiments, pressing an inactivation button configured on the remote control to cease all movements of said first, second and third elongate surgical tools by a robotic device.

In some embodiments, pressing said inactivation button transmits wirelessly a command to said robotic device to perform said cease.

According to an aspect of some embodiments there is provided a method of operation of a remote control for use with a robotic device operable to move first, second and third elongate surgical tools, the method comprising:
receiving, at dedicated right and left button modules of the remote control, commands for controlling movement of the first and second elongate surgical tools;
sending control signals to the robotic device to move the first and second elongate surgical tools; receiving an indication that a tool transition button of the remote control is pressed;
receiving, at one of the dedicated right and left button modules of the remote control, commands for controlling movement of the third elongate surgical tool; and
sending control signals to the robotic device to move the third elongate surgical tool.

According to an aspect of some embodiments there is provided a remote control for use with a robotic device operable to move a plurality of elongate surgical tools, the remote control comprising:
a housing shaped and sized to be held between left and right palms of user;
a plurality of button modules configured on the housing, each button module configured for controlling movement of at least one of the plurality of elongate surgical tools; and
at least two sensors located on the housing at a position of engagement with the user's palms, the at least two sensors configured to detect gripping of the housing between both of the user's palms.

In some embodiments, the plurality of button modules comprise left and right button modules spaced apart from each other on the housing.

In some embodiments, the remote control comprises a tool transition button which, when pressed and held down, switches one of the left and right button modules from control of a first elongate surgical tool to control of a second, different elongate surgical tool.

In some embodiments, the housing is thin and elongate, and wherein a maximal thickness of the housing is lower than 40 mm.

In some embodiments, buttons of the button modules are arranged on the housing within thumb reach when the remote control is held between the two palms of the user.

In some embodiments, the at least two sensors are configured to detect bare hands or hands wearing gloves.

In some embodiments, the at least two sensors include IR sensors.

In some embodiments, an inactivation button configured to cease all movements of said plurality of elongate surgical tools in said robotic device by commanding a software to cut the electrical power of said robotic device.

According to an aspect of some embodiments there is provided a system comprising:
a robotic device operable to move a plurality of elongate surgical tools; and
a remote control configured to control movement of the plurality of elongate surgical tools by the robotic device;
wherein the remote control comprises circuitry programmed to detect an activity level of an imaging modality used with the robotic device, and to allow control of the robotic device via the remote control based on the detected activity level.

According to an aspect of some embodiments there is provided a method of operating a system comprising a robotic device which moves one or more elongate surgical tools, and a remote control through which a user controls movement of the elongate surgical tools by the robotic device, the method comprising:
loading one or more elongate surgical tools onto the robotic device;
positioning the robotic device relative to a body of the patient;
controlling, via the remote control, movement of the one or more elongate surgical tools using the robotic device; and
rendering unusable one or both of the robotic device and the remote control at the end of use.

In some embodiments, the method comprises, prior to the controlling, removing the remote control from a sterile package.

In some embodiments, the method comprises disposing the robotic device along with the one or more elongate surgical tools, and the remote control at the end of use.

According to an aspect of some embodiments there is provided a remote control for use with a robotic device operable to move a plurality of elongate surgical tools, the remote control comprising:
an elongate, thin housing shaped and sized to be held between the hands of a user, the housing comprising a plurality of buttons;
a component configured to be removably received within a designated recess defined in the housing, the component configured to enable actuation of the plurality of buttons and for translating the actuation into control signals for controlling the robotic device.

In some embodiments, the component comprises one or more of: electronics, data, powering and circuitry.

In some embodiments, the component is formed as a removable cassette.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A shows a system comprising a robotic device for moving a plurality of elongate surgical tools, and a remote control through which a user controls movement of the elongate surgical tools by the robotic device, according to some embodiments;

FIG. 1B shows a flowchart of the exemplary path of communication between the user, the remote control and the robotic device, according to some embodiments;

FIG. 2 schematically illustrates use of a remote control by a surgeon during an interventional procedure, according to some embodiments;

FIGS. 3A-C are different views of a remote control device, according to some embodiments;

FIG. 4 is flowchart of a method of controlling, using a remote control, movement of a plurality of elongate surgical tools by a robotic device, according to some embodiments;

FIG. 5 schematically illustrates different types of elongate surgical tool movement and the remote control operation buttons for carrying out the movement, according to some embodiments;

FIG. 6 is flowchart of a method for switching an activation mode at the remote control for controlling movement of third elongate surgical tool, according to some embodiments; and

FIG. 7 is an example of a disposable remote control housing for use with a reusable cassette, according to some embodiments.

### DESCRIPTION OF SPECIFIC EMBODIMENTS OF THE INVENTION

The present invention, in some embodiments thereof, relates to a remote control for use with a surgical robotic device, and, more particularly, but not exclusively, to control of multiple (e.g. more than 2) elongate surgical tools moveable by a robotic device via a remote control held and operated by two hands of a user.

A broad aspect of some embodiments relates to remotely controlling movement of multiple (e.g. two, three, four) elongate surgical tools inserted into the body, where movement of the tools is actuated by a robotic device which receives control signals from a remote control device. In some embodiments, the communication between the robotic device and the remote control device is a two-way communication. In some embodiments, exemplary information that is transmitted between the robotic device and the remote control device are one or more of battery life, movements of parts in the robotic device, movements of tools actuated by the robotic device, notification of malfunctions, notification of status of the tools (for example reaching a range limit of the tool's movement span). In some embodiments, communication of information is performed in a non-graphical manner, meaning, there is no graphical interface unit that shows these exemplary communications. In some embodiments, communication of information to the user is performed using one or more of lights, sounds and vibrations.

An aspect of some embodiments relates to a user interface in the form of a remote control configured to provide for control of more than two elongate surgical tools (e.g. three tools) using an interface operable by two hands, without requiring the user to move their hands from a resting position against the remote control housing when switching from control of first and second tools to control of first and third or second and third tools.

In some embodiments, a remote control is provided with left and right button modules arranged respectively on opposing sides of an elongate housing. In some embodiments, in a default state of the remote control, each of the left and right button modules controls movement of each of first and second tools respectively, for example, movement of a guidewire and a microcatheter. In some embodiments, buttons for moving the tools (of each of the modules) are operated using the left thumb and the right thumb respectively.

In some embodiments, upon a need to control movement of a third tool, for example a guiding catheter, the user presses and holds (e.g. using one of the thumbs) a tool transition button configured on the remote control, which switches at least one of the left and right button modules from control of the default tool to control of the third tool. In some embodiments, the same buttons used for controlling movement of the default first or second tool are now used for controlling movement of the third tool. In some embodiments, the user keeps their hands rested against the remote control housing even during the transition to control of a third tool, and continues operating the remote control using the thumbs. In some embodiments, the buttons protrude slightly outwardly relative to a top surface of the housing, allowing the user to sense their way by tactile motion to the tool transition button and/or the other movement control buttons, potentially without the need to look down at the remote control. This may allow the user to continue viewing, for example, a screen of an imaging modality used during the procedure.

In some embodiments, once the tool transition button is released, the left and right button modules automatically return to control of the first and second tools.

Some embodiments of the invention relate to improving safety when controlling movement of elongate surgical tools actuated by a robotic device.

In some embodiments, the remote control is provided with a stop button, optionally for use in case of emergencies, configured to activate a dedicated software that causes, optionally via wireless means, an activation of a software in the robotic device that generates a software-based electrical shut down of the battery source that provides energy to the motors, thereby causing a complete stop of the motors in the robotic device and ceasing any movement of the tool.

An aspect of some embodiments relates to detection of gripping of a remote control between the two palms of the user's hands. In some embodiments, the remote control comprises a plurality of sensors positioned and configured to detect that the remote control is safely held between two hands of the user. In some embodiments, the sensors are located on opposing side faces of the remote control housing, at locations intended to be covered by the hands of the user when the remote control is properly gripped. In some embodiments, the sensors are located at a position which ensures that the remote control is not simply held by the hands, but rather stably held between the two palms, in manner that ensures the fingers (e.g. thumbs) are in reach of the remote control buttons. In some embodiments, the remote control is operable only when proper gripping is identified.

An aspect of some embodiments relates to a system in which a robotic device is used with an imaging modality, and the remote control of the robotic device is operable only once activation of the imaging modality (for example, initiation of radiation) is identified, and/or when a certain activity level of the imaging modality is identified. For example, the system identifies a rate in which images are obtained, a time that has passed since the last image was obtained, the extent of movement of the tools carried out by the robotic device since the last image was obtained; and based on one or more of these parameters, determine whether control signals should be received at the remote control and issued from the remote control to the robotic device.

A potential advantage of enabling operation of the remote control based on whether an activity level of an imaging modality is active may include reducing or preventing undesired movement of the elongate surgical tools actuated by the robotic device when the user (e.g. surgeon, physician) has no feedback (or no sufficient or recent feedback) regarding the current position, location and/or orientation of the tools inside the body.

An aspect of some embodiments relates to a remote control comprised of single use and multi-use components. In some embodiments, a single use remote control housing is provided, including a shell having a plurality of buttons mounted thereon. In some embodiments, the housing is formed with a recess in which a multi-use cassette that includes electronic components, powering and circuitry is received. In some embodiments, when the cassette is placed in the recess, the buttons are made operable. In some embodiments, the housing (being the component that is directly contacted by the handling user) is disposed following use, while the cassette can be used with another (new) housing. Optionally, the cassette is inserted into a new housing and sterilized along with the packaging of the new housing.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

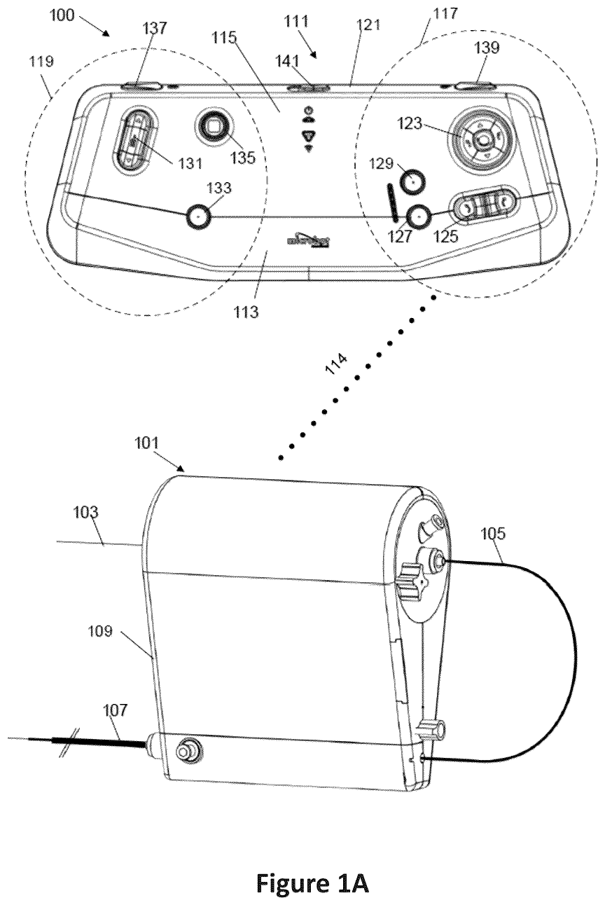

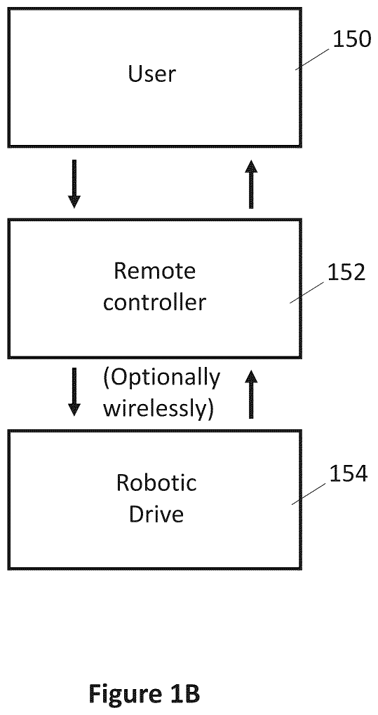

Referring now to the drawings, FIG. 1A shows a system 100 comprising a robotic device 101 for moving a plurality of elongate surgical tools, and a remote control 111 through which a user controls movement of the elongate surgical tools by the robotic device, according to some embodiments.

In some embodiments, a compact robotic device 101 is configured to be loaded (optionally, the device is pre-loaded) with a plurality of elongate surgical tools for use in a procedure, for example an intravascular procedure. Examples of elongate surgical tools which are movable by the device include a guidewire 103; a microcatheter 105; a guiding catheter 107 and/or other. In some embodiments, the tools are configured for telescopic use with each other, for example the guidewire is at least partially received within a lumen of the microcatheter, and the microcatheter (optionally inclusive of the guidewire) is received within a lumen of the guiding catheter.

In some embodiments, a housing 109 of device 101 is shaped and sized to allow positioning of the device in proximity to the patient, for example to the entry point of the tool(s) into the body of the patient. In some embodiments, the device is held adjacent the surgical bed via a mounting arm, optionally adjustable in position. Additionally or alternatively, the device is placed on the surgical bed, on a nearby tray or even on the patient themselves.

In some embodiments, housing 109 accommodates tool movement mechanisms for moving the surgical tools, such as for rolling and/or linearly advancing/retracting a tool. In some embodiments, tool movement mechanisms comprise elements which directly contact the tool, e.g. wheels, and cause its movement. In some embodiments, the tool movement mechanisms are driven by a plurality of motors which are also accommodated within the device housing. Optionally, gear or gear trains transfer the torque from the motor(s) to the tool movement mechanisms (e.g. to the wheels).

In some embodiments, tool movement mechanisms are located relative to the device housing such that they define separate pathways for each of the tools. Optionally, the pathways are parallel to each other. In some embodiments, one or more of the tools forms a curve outside the housing (in this example, see microcatheter 105, inclusive of guidewire 103) when a proximal end of the tool is attached to the housing, optionally externally, while a more distal portion of the tool is received within the designated pathway which passes throughout the housing. In some embodiments, movement of the tool inside the pathway (advancement or retraction) changes the size of the curve outside the housing.

In some embodiments, optionally except for locations of tool attachment, the housing is sealed. In some embodiments, the housing includes a removable or moveable cover or lid providing access to at least one tool residing within the housing. In some embodiments, all components which engage the tool to manipulate it and/or to drive its movement are fully encased inside the inner volume of the housing and at least some of these components are positioned along the pathway defined for the tool.

In some embodiments, each tool is moveable by its own designated tool movement mechanism. In some embodiments, guidewire 103 is configured for to be both rolled and linearly advanced and retracted; microcatheter 105 is configured to be linearly advanced and retracted; and guiding catheter 107 is configured for limited linear translation and for roll. In some embodiments, tool movement mechanisms of different tools are controlled in synchronization so that a tool telescopically received within another tool either moves or remains static with respect to the other tool, depending on the need (e.g. the guidewire is advanced along with the microcatheter).

In some embodiments, manipulation of the tools by the robotic device 101 is controlled remotely, for example via a remote control 111.

In some embodiments, remote control 111 is configured for wireless communication 114 with the robotic device, for example via Wi-fi, Bluetooth, infrared data and/or other wireless protocols.

In some embodiments, in use, a user (e.g. a surgeon, physician or other clinical personnel) holds the remote control between their two hands, and controls movement of the surgical tools by the robotic device by operating buttons and/or other interfaces on the remote control.

In some embodiments, a housing 113 of the remote control is shaped and sized to be ergonomically gripped between the hands (such as between the palms) of the user. In some embodiments, the housing comprises an elongate profile having a substantially rectangular, planar top surface 115.

In some embodiments, housing 113 of the remote control accommodates control circuitry. In some embodiments, housing 113 of the remote control accommodates powering means, for example, a battery for powering the remote control.

In some embodiments, buttons are arranged relative to the housing so as to define two separately engageable modules: a right hand module 117, and a left hand module 119. In use, a user holding the remote control between their two hands rests his fingers on or adjacent the buttons, while the buttons are all within reachable excess of a slight movement of the finger. In some embodiments, buttons located on the top surface are accessed using the thumb (left thumb for left hand module buttons; right thumb for right hand module buttons). In some embodiments, buttons located on a side face 121 of the remote control are accessed using the index or middle fingers.

In some embodiments, the left and right hand modules are sufficiently spaced apart from each other on the housing so that a risk of cross-operation (e.g. the right hand thumb accidentally hitting the left hand module buttons or vice versa) is reduced or prevented. This may allow the user intuitive control without needing to look down at the buttons to select which button to contact. Commonly, the user holds the remote control while at the same time looking at the screen of an imaging module.

In some embodiments, each of the left and right hand modules is configured to control movement of a different surgical tool. For example, the right hand module controls movement of the guidewire, and the left hand module controls movement of the microcatheter. In some embodiments, at least one of the modules can be transitioned to control a different (e.g. third) tool using the same button interfaces, for example, control movement of a guiding catheter. In some embodiments, switching to control of a third tool is carried out by pressing (in some embodiments, pressing and holding down) a tool transition button.

An exemplary remote control button arrangement is described by the following, as demonstrated in FIG. 1A:
In some embodiments, right hand module 117 comprises buttons for controlling movement of a guidewire. In some embodiments, a right thumbstick 123 is configured to actuate: linear advancement (upper arrow); linear retraction (lower arrow); continuous roll to the right (right side arrow); continuous roll to the left (left side arrow). If the thumbstick is pushed in a direction that is in between arrows (i.e. a diagonal direction, midway between the vertical and horizontal axes of the thumbstick), both movements (linear translation and roll) are actuated simultaneously. [For example, pushing diagonally between the upper arrow and the left arrow simultaneously rolls the guidewire to the left and advances the guidewire forwards.

In some embodiments, a 2-way button 125 is configured to actuate discrete (stepwise) roll to the right or to the left.

In some embodiments, separate buttons 127, 129 are configured to move (advance or retract) a guidewire to a known calibration position. For example, button 127 retracts the guidewire, for example to a position in which a sensor (e.g. an optical sensor) senses that the guidewire is no longer within the lumen of the microcatheter. Optionally, the sensor is located adjacent a proximal end of the microcatheter, through which the guidewire exits when fully retracted. In some embodiments, retraction of the guidewire from the microcatheter lumen frees the lumen for example so that injection fluid (e.g. saline, medication) may be introduced through the microcatheter.

In some embodiments, button 129 advances the guidewire, for example to a previous position from which the guidewire was retracted, or to a predetermined distance from that previous position. In some embodiments, the guidewire is automatically advanced to a point that is close to yet still proximal to the previous position, and can be controllably maneuvered (by the user) to the previous position.

In some embodiments, automated guidewire advancement/retraction is carried out based on a count of motor rotations, for example, the number of motor rotations that were required for bringing the guidewire to a position from which it was then retracted, the number of motor rotations required for moving the guidewire between a selected proximal position and a selected more distal position, and/or other absolute or relative positions.

In some embodiments, left hand module 119 comprises button for controlling movement of a microcatheter. In some embodiments, a 2-way left thumbstick 131 controls linear advancement (by pushing the thumstick in the direction of the upper arrow) and retraction (by pushing the thumbstick in the direction of the lower arrow) of the microcatheter. In some embodiments, the left thumbstick is constrained to movement along the vertical axis of the stick only (e.g. by being pushed upwards or downwards).

In some embodiments, the remote control comprises a tool transition button 133 which when pressed and held down (e.g. by the left thumb of the user) switches the mode of control of one of the modules, for example the right hand module buttons to controlling movement of the guiding catheter, instead of the guidewire.

In some embodiments, the tool transition button changes a function of the buttons to make adaptations in the type and/or extent of movement to match the properties of the third tool. In an example, when using the right thumbstick 123 for moving the guidewire, simultaneous roll and advancement/retraction of the guidewire is provided, for example by pushing the right thumbstick in a diagonal direction, which is in between the vertical axis arrows and the horizontal axis arrows. In some embodiments, all tool movements, including simultaneous roll and linear translation, are carried out solely using the thumb of the user.

In some embodiments, when moving the guiding catheter, it may be desired to separate between the advancement/retraction and roll movements, and not to allow them simultaneously. Such separation may be preferred, in some embodiments, for reducing or preventing unintentional rolling of the guiding catheter during linear translation, which could affect a stability of the telescopic tools'arrangement (e.g. microcatheter, guide wire) residing inside the guiding catheter, for example, may dislocate the telescopic arrangement from a current position inside the body. Therefore, in some embodiments, when the tool transition button is activated (e.g. pressed and held down), and the mode is switched to guiding catheter control, the right thumbstick 123 function changes to provide only for advancement/retraction of the guiding catheter, while roll (either continuous or discrete) is enabled solely by the 2-way button 125. In some embodiments, elimination of simultaneous roll and linear translation of the guiding catheter reduces a risk of uni ntentional dislocation of the telescopic arrangement as a whole.

In some embodiments, the change in button function is programmed as a software-based change, which for example neutralizes (or otherwise limits) the control enabled by pressing certain portions of the right thumbstick 123).

In some embodiments, once the tool transition button 133 is released, the right hand module buttons automatically return to guidewire control.

In some embodiments, additional buttons of the remote control include:
An inactivation (stop)button 135 which immediately sends a control signal which ceases all power supply to the motors of the robotic device, thereby stopping movement of all tools; In some embodiments, when the inactivation (stop) button 135 is pressed in the remote control, a dedicated software is activated, which causes sending commands to the robotic device, optionally via wireless means, which causes ceasing all power supply to the motors of the robotic device, thereby stopping movement of all tools.
A dual movement button 137, optionally configured on side face 121, which when pressed (optionally-pressed and held down) moves both of the tools that are currently being controlled by the modules together. For example, the guidewire and microcatheter together; or the microcatheter and guiding catheter together. In some embodiments, when the dual movement button is pressed, the buttons controlling movement of both the tools are the ones of the right hand module. Optionally, either the right or left modules can be used for controlling dual movement of the tools. In some embodiments, the dual movement button 137 functions only when simultaneously used with one of the tool movement buttons engaged by the user's thumb. (For example, the dual movement button 137 is engaged by the index finger, while a button which moves the tool is engaged by the thumb).
A turbo speed button 139, optionally configured on side face 121, which when pressed (optionally-pressed and held down) increases the speed of movement, for example, doubles the speed or otherwise multiplies the existing speed of movement. Optionally, the turbo speed multiplies the default speed by 1.5, 2, 3, 4, 5, or intermediate or larger factor. In some embodiments, the turbo speed button functions only when simultaneously used with one of the tool movement buttons engaged by the user's thumb.
An on/off button 141, optionally configured on side face 121.

Referring now to FIG. 1B, showing a flowchart of the exemplary path of communication between the user, the remote control and the robotic device, according to some embodiments of the invention. In some embodiments, the communication between the user 150, the robotic device 154 and the remote control device 152 is a two-way communication. In some embodiments, data is transmitted from the robotic device 154 to the remote control 152. In some embodiments, data is transmitted from the remote control 152 to the robotic device 154. In some embodiments, communication of data/information is performed wirelessly. In some embodiments, exemplary information that is transmitted/communicated between the robotic device 154 and the remote control device 152, and vice-versa, are one or more of: battery life (of either the remote control 152 or the robotic device 154); movements of parts in the robotic device; movements of tools actuated by the robotic device; movement of controllers/buttons in the remote control; notification of malfunctions, for example vibrations sensed in the robotic device, malfunction of parts in the robotic device, malfunction of buttons in the remote control, etc. ; notification of status of the tools, for example reaching a range limit of the tool's movement span).

In some embodiments, communication of information is performed in a non-graphical manner, meaning, there is no graphical interface unit that shows these exemplary communications. In some embodiments, communication of information to the user 150 is performed using one or more of lights, sounds and vibrations.

In some embodiments, as mentioned above, the remote control 152 and/or the robotic device 154 include visual indications for indicating a current status to the user. For example, the robotic device housing 109 and the remote control housing 113 each include a plurality of light indications, e.g. LEDs.

In an example, the robotic device is configured to indicate, via the light indications (e.g. LEDs on the housing): that the device is powered on; low power (e.g. low battery); that there is proper communication between the remote control and the robotic device; that the device is in initialization mode; that tool(s) are currently being moved by the device; a direction of tool movement (e.g. advancement/retraction); that an error has occurred; and/or others.

In an example, the remote control is configured to indicate, via the light indications (e.g. LEDs on the housing): that the remote control is powered on; low power (e.g. low battery); that there is proper communication between the remote control and the robotic device; that buttons of the remote control are currently being pressed; that the tool transition button is pressed, changing the mode of control to a third tool; that the remote control is in initialization mode; a direction of tool movement (e.g. advancement/retraction); that an error has occurred; and/or others.

It is noted that in some embodiments indications may be other than visual, for example, the remote control may provide tactile indication (e.g. via vibration), audible indications, and/or other.

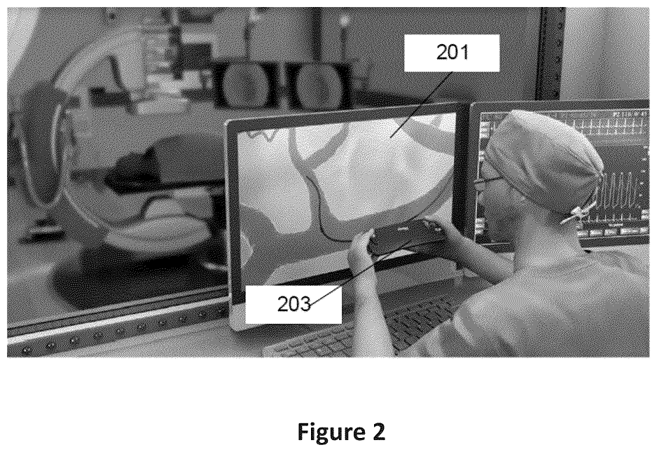

FIG. 2 schematically illustrates use of a remote control by a surgeon during an interventional procedure, according to some embodiments.

In some embodiments, insertion and/or navigation and/or other manipulation of the surgical tools inside the body is performed under imaging, for example under CT, MRI, fluoroscopy, ultrasound and/or other imaging modality.

In some embodiments, the user (e.g. surgeon) is situated away from the surgical bed, optionally in a different room. During the procedure, the user views the results of real time imaging on the screen 201 while at the same time holding the remote control 203 between their hands and operating the remote control buttons.

In some embodiments, as a safety measure, the remote control is activated only when imaging is activated (e.g. radiation is turned on). This may reduce or prevent a situation in which the user moves the surgical tools using the remote control without the ability to view the tools'specific anatomical location and position.

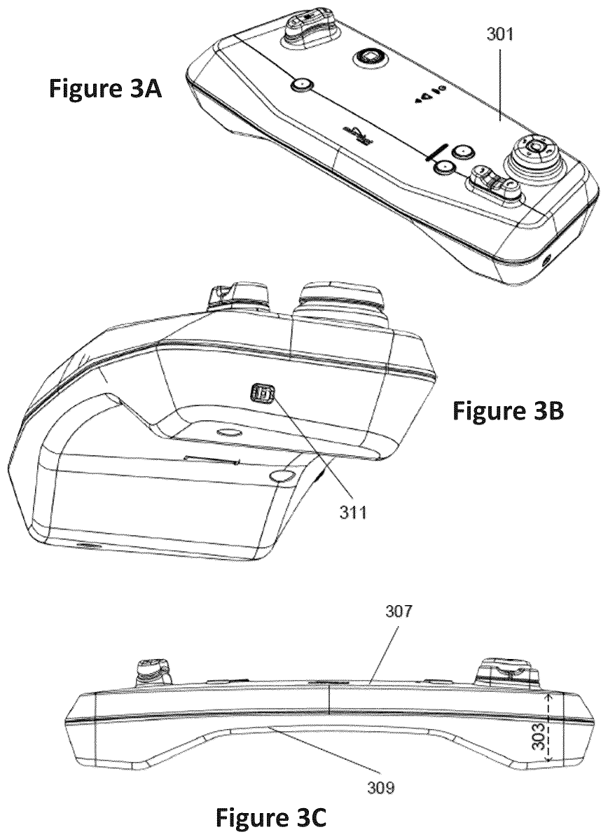

FIGS. 3A-C are different views of a remote control device, according to some embodiments.

In some embodiments, the remote control 301 is shaped and sized to be gripped between the palms of the user's hands. In some embodiments, a maximal thickness 303 (see FIG. 3C) of the remote control housing 305 is small enough to fit into palms of various sizes, for example the maximal thickness is smaller than 50 mm, smaller than 40 mm, smaller than 30 mm, or intermediate, higher or lower thickness.

In some embodiments, a top surface 307 of the housing is substantially flat, while a bottom surface 309 of the housing is formed with a curve (or bridge shape). A potential advantage of a curvy bottom surface may include facilitating gripping of the remote by the user's palms.

In some embodiments, one or more of the remote control buttons protrude externally from the top surface 307, for example to a distance of 0.5-10 mm, 10-20mm, 1-15 mm or intermediate, larger or shorter distance. In some embodiments, press buttons (for example the automated retraction/advancement buttons) protrude to an extent shorter than that of the thumbstick. A potential advantage of the buttons protruding from the surface may include facilitating sensing of a button location and/or facilitating engagement of a user's finger (e.g. thumb) with the button surface, without having the need to look at the controller. A potential advantage of a height difference between the different button types may include a further improved sensible control of the specific type of button being currently engaged by the user.

In some embodiments, the remote control comprises means for detecting presence of the user's hands, for example, a plurality of sensors 311 configured for detecting that the remote control is now held between the two hands of a user. In some embodiments, sensors 311 are located on opposing side faces of the housing, at a position which, when the remote control is gripped by the user, is covered by the palms of the hands. In some embodiments, sensors 311 are light based sensors, e.g. IR sensors. In some embodiments, light emitted from the sensors is reflected back from the user's hands when the remote control is gripped by the hands.

In some embodiments, only upon detection of presence of the hands, remote control operation is enabled. In an example, only upon detection of presence of the hands, electrical power supply to the remote control buttons is enabled. In some embodiments, if loss of hand presence is detected, for example if the user accidentally drops the remote control or even releases it from one of the hands, remote control operation is ceased.

In some embodiments, sensors 311 are configured to detect both bare hands (the user's skin) as well as hands covered by gloves.

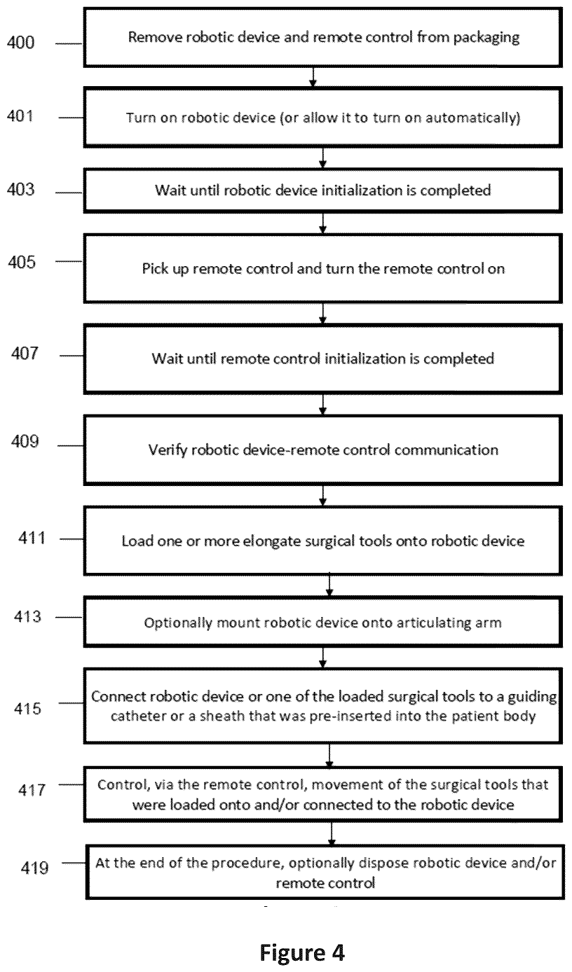

FIG. 4 is flowchart of a method of controlling, using a remote control, movement of a plurality of elongate surgical tools by a robotic device, according to some embodiments.

In some embodiments, during setup of the system, a user (e.g. surgeon, surgical room nurse, physician, technician or other clinical personnel) removes the robotic device and the remote control from their sterile packaging (400).

In some embodiments, the user turns on the robotic device (401), such as by pressing an on/off button, removing a battery protection slip. Alternatively, the robotic device turns on automatically, for example when removed from its sterile packaging, when mounted onto a mounting arm and/or other platform, when loaded with tools, and/or other.

In some embodiments, the user waits for an indication (e.g. a light based indication) that device initialization is completed (403).

In some embodiments, a user which is intended to operate the remote control (e.g. a surgeon, physician) picks up the remote control between their hands, and turns the remote control on (405). In some embodiments, the user waits for completion of remote control initialization (407). In some embodiments, the user verifies communication between the remote control and the robotic device (409), as indicated for example by a light based indication on both devices.

In some embodiments, one or more surgical tools are loaded on to the robotic device (411). In some embodiments, loading comprises attaching a proximal end of a tool to the robotic device housing, and inserting a more distal portion of the tool into a designated aperture leading to the inner volume of the device. In an exemplary construction, a guidewire and a microcatheter are loaded onto the device, with the guidewire extending at least in part into a lumen of the microcatheter. In some embodiments, each of the tools is loaded and received within its own designate pathway in the robotic device housing. Additionally or alternatively, one or more of the tools arrives pre-loaded in the device, for example, a guidewire may be contained in the device so that when the device is removed from its packaging, the guidewire is already loaded into its designated pathway and ready to use.

In some embodiments, optionally, the robotic device is mounted onto an articulating arm (413). In some embodiments, the articulating arm is adjusted to hold the robotic device at a selected position (e.g. at a selected distance from the surgical bed or patient; at a selected angle relative to the surgical bed or patient; and/or other selected position). In some embodiments, the articulating arm is configured to hold the robotic device in close proximity to an entry point into the patient body.

Additionally or alternatively, the robotic device is placed on the surgical bed, on an adjacent platform, or optionally on the patient themselves.

In some embodiments, the robotic device and/or one or more of the tools that were loaded on it are connected (for example, the tools are advanced into a lumen) of a guiding catheter or a sheath that had been pre-introduced into the patient body (415). In an example, a proximal end of the guiding catheter is attached to a designated coupling (e.g. a luer) on the robotic device housing, and the microcatheter (optionally with the guidewire extending at least partially therein) is advanced, by the robotic device, into the guiding catheter lumen.

In some embodiments, once the robotic device is assembled and ready for use and the user has picked up the remote control in their hands, the user controls, via the remote control buttons, movement of the tools that were loaded onto and/or connected to the robotic device (417). In some embodiments, control of movement comprises control of: roll of the tool (i.e. about the tool long axis); linear advancement or retraction of the tool; simultaneous roll and linear movement of the tool; control of the tool speed of movement; control of synergistic movement of at least two tools. In some embodiments, the types of movements and/or extent of movements are defined specifically per the tool to be controlled. For example, the right hand module buttons are programmed to actuate guidewire movement, which, in some embodiments, is less limited than microcatheter movement, controlled by the left hand module buttons. (For example, while the guidewire can be both rolled and linearly moved, the microcatheter is not allowed to roll).

In some embodiments, at the end of the procedure and after the tools were fully retracted from the patient body, the robotic device and/or the remote control are disposed (419). In some embodiments, the robotic device is disposed along with the tools. Optionally, both the robotic device and the remote control are disposed. In some embodiments, the robotic device and/or the remote control are thrown away, destroyed and/or otherwise rendered unusuable. A potential advantage of a single use system may include reducing or avoiding the need for sterilization processes following operation; reducing or avoiding a risk of contamination to the patient (as in some embodiments the system arrives in a sterilized package), and facilitating handling of the system, for example as no draping is required.

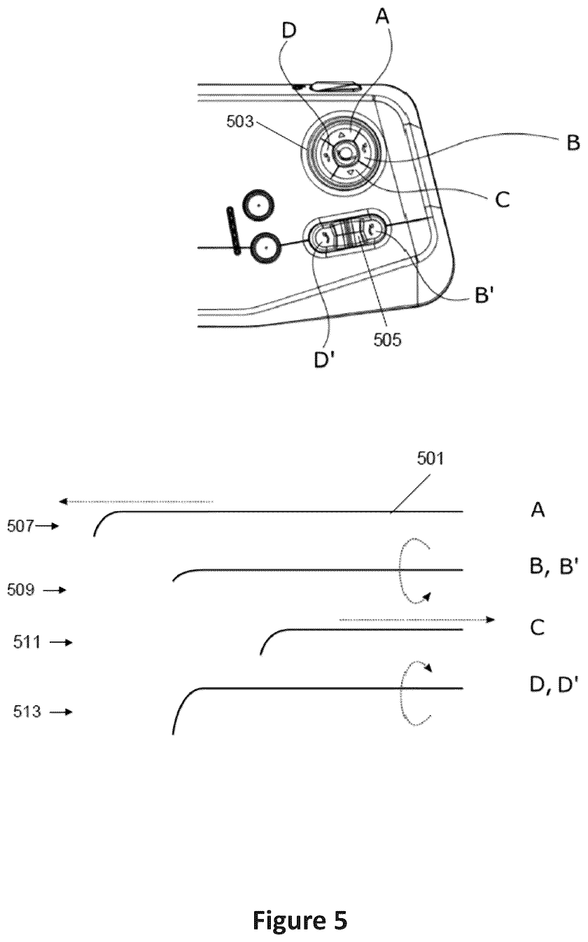

FIG. 5 schematically illustrates different types of elongate surgical tool movement and the remote control operation buttons for carrying out the movement, according to some embodiments.

The example of FIG. 5 shows control of movement of a guidewire 501. In some embodiments, the following movements are carried out using the right thumbstick 503 and the 2-way button 505, for example by placing the right hand thumb on the these buttons:
In some embodiments, forward linear movement is carried out by pushing the right thumbstick 503 in the direction of “A” ; backward linear movement is carried out by pushing the right thumbstick in the direction of “C”; continuous roll movement to the right is carried out by pushing the right thumbstick 503 in the direction of “B”; continuous roll movement to the left is carried out by pushing the right thumbstick 503 in the direction of “D” o; discrete (step-wise) roll movement to the right is carried out by pressing arrow “B”” on the 2-way button 505; and discrete (step-wise) roll movement to the left is carried out by pressing arrow “D” on the 2-way button 505.

Examples of guidewire movements carried out by the above described actuation are shown at the bottom of FIG. 5: at 507, guidewire 501, (in this example having a curved distal tip), is advanced forward; at 509, guidewire 501 is rolled to the right (either continuously or discretely); at 511, guidewire 501 is retracted backwards; at 513, guidewire 501 is rolled to the left (either continuously or discretely).

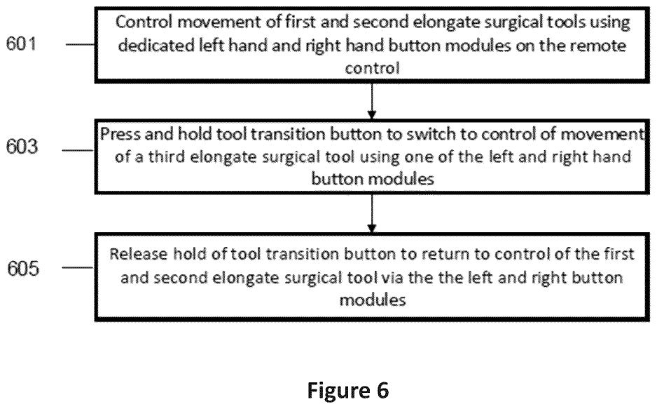

FIG. 6 is flowchart of a method for switching an activation mode at the remote control for controlling movement of a third elongate surgical tool, according to some embodiments.

In some embodiments, when three different elongate surgical tools are manipulated by the robotic device, the remote control is configured to enable fast switching of a control mode for using the same button interface that was used for controlling one of the first and second tools, to control of the third tool. In this manner, the user may continue holding the remote control, resting their thumbs on the top surface of the remote control, and simply switch to control of the third tool by pressing the tool transition button.

In an exemplary method of use, the user controls movement of first and second elongate surgical tools using the dedicated left and right hand button modules on the remote control (601). In an example, the first and second tools include a guidewire and a microcatheter.

When it is required to move a third tool, for example, linearly move and/or roll a guiding catheter, the user presses and holds the tool transition button to switch to control of movement of the third tool using one of the of the left and right hand button modules (603). In an exemplary configuration, the right hand module serves, as default mode, to control the guidewire, and upon pressing the tool transition button, the right hand module serves to control the guiding catheter.

In some embodiments, when the user wants to return to control of the guidewire and/or when there is no longer a need to move the guiding catheter, the user releases hold of the tool transition button and returns to the default control of the guidewire (605). In some embodiments, the left hand button module remains dedicated to control of the microcatheter at all operational modes.

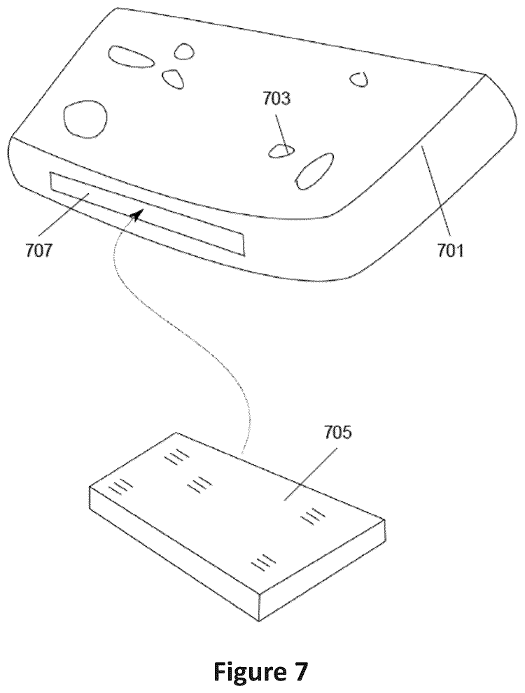

FIG. 7 is an example of a disposable remote control housing for use with a reusable cassette, according to some embodiments.

In some embodiments, the remote control is comprised of a disposable, single use housing 701, which includes an external shell with buttons 703, and is configured to receive therein a reusable cassette 705 which includes electronics, power and circuitry required for making the remote control operable. In some embodiments, housing 701 is formed with a recess 707 shaped and sized for receiving the cassette therein.

In some embodiments, before use, the housing is removed from a sterilized package, and the cassette is inserted into the designated recess. Following the procedure, the cassette is removed from the housing. The housing (which was directly contacted by a user) is disposed, and the cassette can be sterilized for additional use with a new housing. Optionally, a used cassette is inserted into a new housing during packaging of the housing, and the cassette and housing are sterilized together. Potential advantages of a reusable cassette and a disposable remote control housing may include reduced manufacturing costs, easier disposal and potentially recycling of the housing, and others.

In some embodiments, different cassettes may be provided for setting different sets of functions to the same buttons on the housing. Optionally, a user (e.g. surgeon, physician) selects a cassette which sets specific button functions that would be most commonly used during a specific procedure.

The terms “comprises”, “comprising”, “includes”, “including”, “having” and their conjugates mean “including but not limited to”.

The term “consisting of”′ means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of controlling, using a remote control, movement of first, second and third elongate surgical tools by a robotic device, the method comprising:
controlling movement of said first and second elongate surgical tools via dedicated right and left button modules configured on the remote control;
continuously holding a tool transition button configured on the remote control to control movement of said third elongate surgical tool using one of the right or left button modules; and
releasing said tool transition button to return to control of said first and second elongate surgical tools via the right and left button modules.

2. (canceled)

3. The method according to claim 1, wherein said right and left button modules control movement of said first and second elongate surgical tools by default.

4. (canceled)

5. The method according to claim 1, wherein said controlling movement comprises one or both of rolling said first and second elongate surgical tools, and linearly advancing or retracting said first and second elongate surgical tools.

6. The method according to claim 5, wherein said controlling movement comprises rolling and linearly advancing or retracting said first and second elongate surgical tools simultaneously, using the same control button on said remote control.

7. The method according to claim 1, wherein said controlling movement comprises increasing a speed of movement said first and second elongate surgical tools.

8. The method according to claim 1, wherein said controlling movement comprises simultaneously moving two of said first, second and third elongate surgical tools.

9. The method according to claim 1, further comprising:
a. holding the remote control between the left and right palms of the user and operating said right and left button modules using the thumbs; and
b. detecting gripping of the remote control between both of the user's palms, and allowing said controlling only if gripping is detected.

10. The method according to claim 1, further comprising viewing a screen of an imaging modality while at the same time controlling said remote control.

11. The method according to claim 1, wherein said controlling movement via said remote control is performed at a different room than the surgical room in which the robotic device is located.

12. The method according to claim 1, wherein when said tool transition button is continuously held, a function of at least some of said buttons of said right or left module changes.

13. The method according to claim 1, wherein when said tool transition button is continuously held, an extent of movement enabled by at least some of said buttons of said right or left module changes.

14. The method according to claim 1, further comprising pressing an inactivation button configured on the remote control to cease all movements of said first, second and third elongate surgical tools by a robotic device;
and wherein said pressing said inactivation button transmits wirelessly a command to said robotic device to perform said cease.

15. (canceled)

16. The method according to claim 1, further comprising:
a. sending control signals to said robotic device to move said first and second elongate surgical tools;
b. receiving an indication that a tool transition button of the remote control is pressed;
c. receiving, at one of said dedicated right and left button modules of said remote control, commands for controlling movement of said third elongate surgical tool; and
d. sending control signals to said robotic device to move said third elongate surgical tool.

17. A remote control for use with a robotic device operable to move a plurality of elongate surgical tools, the remote control comprising:
a housing shaped and sized to be held between left and right palms of user;
a plurality of button modules configured on said housing, each button module configured for controlling movement of at least one of said plurality of elongate surgical tools; and
at least two sensors located on the housing at a position of engagement with the user's palms, the at least two sensors configured to detect gripping of the housing between both of the user's palms;
wherein said at least two sensors are configured to detect bare hands or hands wearing gloves.

18. The remote control according to claim 17, wherein said plurality of button modules comprise one or more of:
a. left and right button modules spaced apart from each other on said housing; and
b. a tool transition button which, when pressed and held down, switches one of said left and right button modules from control of a first elongate surgical tool to control of a second, different elongate surgical tool.

19. (canceled)

20. The remote control according to claim 17, wherein said housing is thin and elongate, and wherein a maximal thickness of said housing is lower than 40 mm; and wherein buttons of said button modules are arranged on said housing within thumb reach when the remote control is held between the two palms of the user.

21-23. (canceled)

24. The remote control according to claim 17, further comprising an inactivation button configured to cease all movements of said plurality of elongate surgical tools in said robotic device by commanding a software to cut the electrical power of said robotic device.

25-28. (canceled)

29. The remote control according to claim 17, further comprising
a component configured to be removably received within a designated recess defined in said housing, said component configured to enable actuation of said plurality of buttons and for translating said actuation into control signals for controlling said robotic device.

30. The remote control according to claim 29, wherein said component comprises one or more of: electronics, data, powering and circuitry.

31. The remote control according to claim 29, wherein said component is formed as a removable cassette.
